(19) United States
(12) Patent Application Publication
(10) Pub. No.: US 2025/0098058 A1
(43) Pub. Date: Mar. 20, 2025

(54) ANNULAR APPARATUS FOR GENERATING ACCELERATED ELECTRONS

(71) Applicant: Fraunhofer-Gesellschaft zur Förderung der angewandten Forschung e.V., Munich (DE)

(72) Inventors: Gösta MATTAUSCH, Dresden (DE); Jörg KUBUSCH, Dresden (DE); Burkhard ZIMMERMANN, Dresden (DE); Ralf BLÜTHNER, Dresden (DE); Lars DINCKLAGE, Dresden (DE); Severin DOMINOK, Dresden (DE); Henrik FLASKE, Dresden (DE); Ludwig LORENZ, Dresden (DE); Björn MEYER, Dresden (DE); Michiel Top, Dresden (DE)

(73) Assignee: Fraunhofer-Gesellschaft zur Förderung der angewandten Forschung e.V., Munich (DE)

(21) Appl. No.: 18/969,157

(22) Filed: Dec. 4, 2024

Related U.S. Application Data
(63) Continuation of application No. PCT/EP2023/062031, filed on May 5, 2023.

(30) Foreign Application Priority Data
Jun. 8, 2022 (DE) 10 2022 114 434.8

(51) Int. Cl. H05H 7/08; H05H 1/24; H05H 1/54

(52) U.S. Cl. CPC H05H 7/08; H05H 1/4697; H05H 1/54; H05H 2007/084; H05H 2245/36

(57) ABSTRACT

An annular apparatus is provided for generating accelerated electrons, wherein ions from a glow discharge plasma may be accelerated onto the surface of an annular second cathode and electrons emitted by the annular second cathode may be accelerated towards an annular electron exit window by a second electrical voltage applied between the annular second cathode and an annular second anode, wherein a housing is designed as a first cathode; a first anode comprises a number of wire-like electrodes which extend completely or partially through an annular evacuable space, and wherein a second reservoir contains a hydrocarbon-containing compound which may be admitted into the evacuable space through the at least one first inlet.

### CROSS-REFERENCE TO RELATED APPLICATIONS

This application is a continuation of PCT/EP2023/062031 filed May 5, 2023, which claims priority under 35 USC § 119 to German patent application 10 2022 114 434.8 filed Jun. 8, 2022. The entire contents of each of the above-identified applications are hereby incorporated by reference.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 shows a schematic and perspective sectional view of an annular apparatus according to the invention;

FIG. 2 shows a schematic representation of the annular apparatus from FIG. 1 in a plan view;

FIG. 3 shows a schematic sectional view of a first alternative apparatus according to the invention;

FIG. 4 shows a schematic sectional view of an alternative protective screen;

FIG. 5 shows a schematic sectional view of a second alternative apparatus according to the invention; and

FIG. 6 shows a schematic exploded view of a rotationally symmetrical element from FIG. 5.

### DETAILED DESCRIPTION

The invention relates to an annular apparatus for generating accelerated electrons. In particular, an apparatus according to the invention may be used for subjecting bulk materials, extrusion materials and fluids to accelerated electrons. The preferred field of application is phytosanitary treatment of seeds for seed-borne harmful organisms, which are predominantly located in the seed coat of the seeds. Further fields of application are sterilization and modification of the near-surface layer of granulates and powders, e.g., also of animal feed, herbs and spices, chemical surface activation of recyclates and implementation of other radiation-chemical and plasma-chemical processes.

Various methods and the corresponding apparatuses for subjecting bulk materials and extrusion materials to accelerated electrons are known in various designs adapted to the bulk material or extrusion material to be treated.

For example, an electron field with opposing electron velocity components is generated in an evacuated chamber by arranging two electron accelerators opposite one another, through which the bulk material is guided in free fall in an extended transparent stream (DD 291 702 A5). For electron treatment, the bulk material is fed into the chamber via rotary feeders and then discharged following the electron treatment process. The disadvantage of such apparatuses, however, is the great complexity of the equipment for generating the electron field, since at least two electron accelerators with corresponding high-voltage power supplies are required, as well as the great complexity of the vacuum technology.

Also known is generating an electron field with opposing velocity components by redirecting the electron beam, after it has passed through the stream of bulk material particles, back to the particle stream using magnetic deflection. Apparatuses of this type avoid the complexity of a second electron accelerator. The disadvantage of this method, however, is that the relatively long path the electron beam travels in the process chamber requires a substantially better vacuum, which requires even greater equipment complexity to generate the vacuum.

Methods and apparatuses are also known that operate with two electron accelerators facing each other, wherein the electrons exit via a beam exit window at atmospheric pressure (DE 44 34 767 C1). Here too, the bulk material is guided through the electron field in free fall. This solution eliminates the complexity required to evacuate the process chamber. Nevertheless, the high degree of equipment complexity remains due to the required use of at least two electron accelerators.

Also known is subjecting powdered and granular materials at atmospheric pressure to electrons, where only one electron accelerator is used and the particles to be irradiated are carried through the electron field in a gas stream (WO 98/43274 A1). The gas stream with the particles to be irradiated is guided through a rectangular channel, closed on one side with a 25 μm thick aluminum film, into which the electrons penetrate after being discharged via a 13 μm thick titanium window film and passing through the distance to the irradiation channel. Opposite the aluminum film, the rectangular channel is formed using a flat plate made of a material with a high atomic number. After penetrating the channel cross-section, the electrons are backscattered to a certain extent by this plate. The backscattered electrons have a velocity component directed counter to the original direction of incidence of the electrons and permit the side of the particles facing away from the original direction of incidence of the electrons to be exposed to electron bombardment, as well.

The disadvantage is that the intensity of the irradiation by the backscattered electrons is significantly lower than the intensity of the irradiation by the electrons directly exiting the beam exit window, which leads to uneven irradiation of the individual particles. Another disadvantage is that the gas velocity required for carrying the particles increases sharply as the ratio of mass to surface area of the transported particles increases. Thus, very high gas flow velocities would be required for larger-grained bulk materials such as, e.g., wheat or corn. At these high velocities, the energy doses that may be transmitted in the electron field would be limited to very small values that are far too low for many applications. A further disadvantage of this known solution is that after exiting the electron accelerator, the electrons must also penetrate the aluminum film that closes the rectangular channel before they strike the particles to be treated. As a result, the electrons suffer an additional undesirable loss of energy.

Furthermore, DE 199 42 142 A1 describes an apparatus in which bulk material is guided past an electron beam device in a plurality of free falls and is subjected to accelerated electrons. Due to the plurality of passes, combined with the interim mixing of the bulk material, the probability that the particles of the bulk material are subjected to accelerated electrons on all sides is very high in this embodiment. However, the plurality of passes requires a lot of time to carry out the treatment process.

DE 10 2013 111 650 B3 and DE 10 2013 113 688 B3 disclose apparatuses in which an annular electron beam source is designed such that electrons emitted and accelerated by an annular cathode exit from an electron exit window towards the ring axis. The annular electron beam generator is arranged so that its ring axis is oriented as perpendicularly as possible. Arranged above the annular electron beam generator is a device for separating bulk material particles, and the floor of said device has at least one opening out of which bulk material particles fall and travel from there through the ring formed by the electron beam generator. An advantage of such an annular apparatus is that it is more compact than apparatuses consisting of two planar beam sources. However, the disadvantage still is that the minimum velocity at which the bulk particles are guided past an electron exit window is determined by gravitational acceleration and it is not possible for this minimum velocity to be any lower. Therefore, such an apparatus requires relatively high-power electron beam generators that make it possible for the required electron dose to be applied in the short time within which the bulk material particles pass by an electron exit window. Another disadvantage is that the velocity of the bulk material particles increases continuously as said particles pass the electron exit window, which means that as a bulk material particle passes the electron exit window, it is subjected to a continuously decreasing electron density.

A special feature of the annular electron beam generators described in DE 10 2013 111 650 B3 is that the beam electrons are not thermally emitted by heating the cathode (“hot cathode”), but are knocked out of it by ion impact (“cold cathode”). The ions required for this are generated by gas discharges in a plasma chamber, which is also annular and is arranged between the annular cold cathode and the annular electron exit window. For reasons of high-voltage insulation, these must burn at particularly low pressures, which requires special discharge arrangements.

Furthermore, EP 3 590 125 B1 discloses annular electron beam generators having a cold cathode which use a wire anode arrangement for ion production, where the annular plasma space is divided into annular segments by means of walls and where at least one wire-like anode extends through each annular segment for forming a plasma in the annular segment. In EP 3 642 861 B1, this principle of plasma generation in individual segments is used to construct an electron beam generator in which the ring is incomplete, that is, has a permanently open sector, or consists of two half-rings that may be separated and fitted back together without breaking the vacuum.

The described segmentation of the plasma space has a disadvantageous effect because the walls have the same electrical voltage potential as the cathodes of the gas discharge (which is described in detail and clearly in the exemplary embodiment as being identical to that of the housing and the electron exit window, that is, the ground potential). As a result, the cathode fall of the discharge and a lower plasma density are formed in their vicinity, so that a lower ion current density may be extracted there than from the center of the annular segments and accelerated towards the cold cathode, and thus it is also impossible to provide a uniform electron current density and irradiation intensity in the entire ring circumference.

Electron beam generators having a cold cathode have numerous advantages that are set out in the documents cited above, but in practical use, especially in continuous operation, they prove to be problematic from the point of view of operational stability.

An important criterion for the operational stability of electron beam sources is the so-called dielectric strength. The frequency of high-voltage flashovers is usually used as a measure of the dielectric strength of electron sources. Not only do flashovers interrupt beam generation, they also wear out the built-in elements of the source and strain the semiconductor electronics of the control and power supply devices. The flashover rate must therefore be reduced to a tolerable level using time-consuming conditioning routines before the actual beam operation begins. The situation is made more difficult by the high parasitic capacitance of the necessarily large-area cold cathodes, which in the event of a flashover, may lead to HF oscillations and resulting AC loads and destructive overvoltages in the assembly system (high-voltage cables, connectors and bushings). Furthermore, it should be noted that the initial conditioning in beam operation is not effective over a long period. The flashover rate then increases again, largely fueled by a further mechanism: the depositing of insulating coatings on the critical cathode-insulator transition (“triple point”). These insulating near-surface layers adjacent to the active emission zone are formed by the ion impact in the center of the cold cathode, sputtering of aluminum atoms as well as their backscattering and oxidation (due to oxygen in the residual gas and adsorbed water films), creating aluminum (hydr)oxide. Charge carriers may accumulate thereon and produce voltage differences along the surface, said differences being balanced out from time to time by creeping sparks. The latter are harmless in and of themselves, but act as ignition sources for high-voltage flashovers (high-current arc discharges) from the cathode to the ground. These cause a roughening of the cathode and a further increase in the tendency to flashover with increasing operating time due to field emission from sharp-edged microstructures (such as “crater edges,” peaks, splashed melt droplets).

The dose applied to the material to be treated is determined not only by the energy of the electrons (which may be easily controlled by the accelerating voltage) but also by their current density (which is subject to some degree of uncertainty). This uncertainty arises from the fact that the emission current density of the secondary electrons triggered by the ion bombardment of the cold cathode is, in large measure, a function of the chemical and morphological nature of the cathode surface. Fewer or more secondary electrons are released per projectile ion depending on whether the surface is bare of metal or covered with a thin oxide layer. The ratio of the two species is called Townsend's second ionization coefficient and is usually abbreviated as “γ” in the technical literature. It may change over time, as is well known from the literature, based on ion bombardment (and resulting sputter removal of coatings and near-surface layers of the cathode), desorption of water, varying oxygen partial pressure (and growth of dielectric compound layers on the cathode surface controlled thereby). Less extensively investigated and documented in the literature, but plausible, are influences of surface roughness and crystallite structure, and thus of the semi-finished product quality and the aging state of the cold cathode over the operating period.

However, the efficiency of beam generation depends on γ, that is, how much electron current (out of the cathode) is created by how much cathode current (supplied to the cathode from the high-voltage device). This ratio (and thus ultimately the beam current) is difficult to determine using measurements, since the cathode current (easily measurable in the high-voltage device) not only compensates for the amount of charge of the emitted negative electrons, but also, indistinguishably, for the amount of charge of the absorbed positive ions.

In summary, it may be stated that
1) the beam current is monotonically a function of the cathode current, but its exact value is unknown if the coefficient γ is not known, and
2) changes in the coefficient γ that cannot be directly measured result in unnoticed changes in the beam current despite a constant cathode current, and
3) local variations in the coefficient γ translate directly to variations in the local electron current density, which cannot be determined either.

These are fundamental weaknesses of cold cathodes, which become increasingly apparent as the cathode area is scaled up.

A further serious problem is the tendency of the wire anode plasma, which produces the ions required to release the secondary electrons from the cathode, to localize. From a technology point of view, the aim is often to achieve the best possible (rotationally symmetrical) homogeneity of the plasma density in order to achieve a homogeneous current density of the ions striking the cathode and thus also of the secondary electrons emitted by it along the circumference. However, in the case of small discharge currents (such as those required to regulate small electron currents) and low pressures (which are conducive to stable insulation of the high voltage), a wire anode discharge tends to no longer burn evenly across the entire circumference of the source, but instead only in the vicinity of a few wires. Accordingly, ion and electron currents are also concentrated in this sector. In addition to a treatment dose that is unevenly distributed across the circumference of the source, this condition may also lead to destruction of the electron exit window due to local thermal overload of the titanium film, since the beam current at its full programmed height strikes a much smaller area of the electron exit window.

Ultimately, it is precisely these interfering effects that are to be counteracted by the above-cited documents EP 3 590 125 B1 and EP 3 642 861 B1 by segmenting the plasma space, but at the cost of systematically uneven plasma, ion and electron current densities across the circumference caused by the walls of the segments, and thus also at the cost of the treatment dose.

The underlying technical problem of the invention is therefore to create an apparatus for generating accelerated electrons, by means of which the disadvantages of the prior art may be overcome. In particular, the apparatus according to the invention is intended to further develop annular electron beam sources such that cold cathode sources also remain operationally stable over long periods, the material to be treated (bulk material particles, extrusion material or fluids) is subjected to an electron dose that is well defined temporally and across its entire circumference (the electron dose being homogeneous or adjustable as a function of the angle depending on the material to be treated and technology requirements) while passing an electron exit window, dirt particles are kept away from the electron exit window and the complexity required for cooling the system is reduced while still permitting a high throughput of material to be treated.

An apparatus according to the invention includes an annular electron beam generator in which the electrons emitted and accelerated by an annular cathode exit from an annular electron exit window in the direction of the ring axis. The electron exit window is thus at least a component of the annular inner wall of the annular electron beam generator and has the shape of a hollow cylinder.

At this point, it should be expressly pointed out that, for all annular apparatuses, components and hollow cylinders described below, the term “annular” in the context of the invention is not limited to a ring in a circular shape, but instead the term “annular” in the context of the invention refers only to a loop-shaped, closed object, where the cross-section of the loop-shaped, closed object completely encloses a volume and where objects to be treated with accelerated electrons may be guided through this volume in the ring interior. In a preferred embodiment of the invention, the cross-section of the volume completely enclosed by a ring or a hollow cylinder is circular, but may also have any other geometric shape in the broadest sense of the invention.

Such an annular electron beam generator includes an annular housing which delimits an annular evacuable space and has an annular electron exit window; a first reservoir containing a working gas; at least one first inlet for supplying the working gas from the first reservoir into the annular evacuable space; at least one first cathode and at least one first anode, between which a glow discharge plasma may be generated in the annular evacuable space by means of a first applied electrical voltage, where ions from the glow discharge plasma may be accelerated onto the surface of an annular second cathode and electrons emitted by the annular second cathode may be accelerated towards the annular electron exit window by means of a second electrical voltage applied between the annular second cathode and an annular second anode. In an apparatus according to the invention, the housing is further designed as a first cathode and the first anode includes a number of wire-like electrodes which extend completely or partially through the annular evacuable space. An apparatus according to the invention further includes a second reservoir containing a hydrocarbon-containing compound which may be admitted into the annular evacuable space through the at least one first inlet.

In an embodiment of the invention, the ring axis of the annular housing is oriented perpendicularly or at an angle of up to 10° from the perpendicular, so that, for example, bulk material to be treated with accelerated electrons may fall from above to below through the annular opening of the annular housing. A protective screen which mechanically protects the electron exit window from falling bulk material particles may be arranged within the annular opening.

In a further embodiment, the apparatus according to the invention has a first device with which a flow of a gaseous medium (such as air, for example) directed from below to above may be formed within the annular opening (that is, within the volume enclosed by the electron exit window) and counteracts the free fall of the bulk material particles and slows the fall of the bulk material particles. This makes it possible to increase the mean dwell time of the bulk material particles in front of the electron exit window and, as a result, to use an electron beam generator with lower power in order to achieve the same irradiation result as with prior art electron beam generators in which bulk material particles are guided past an electron exit window in a free fall.

Such an upwardly directed flow of a gaseous medium may be formed, for example, if the gaseous medium is blown from below by means of the first device into the intermediate space between the electron exit window and the protective screen.

The invention is explained in greater detail below using exemplary embodiments.

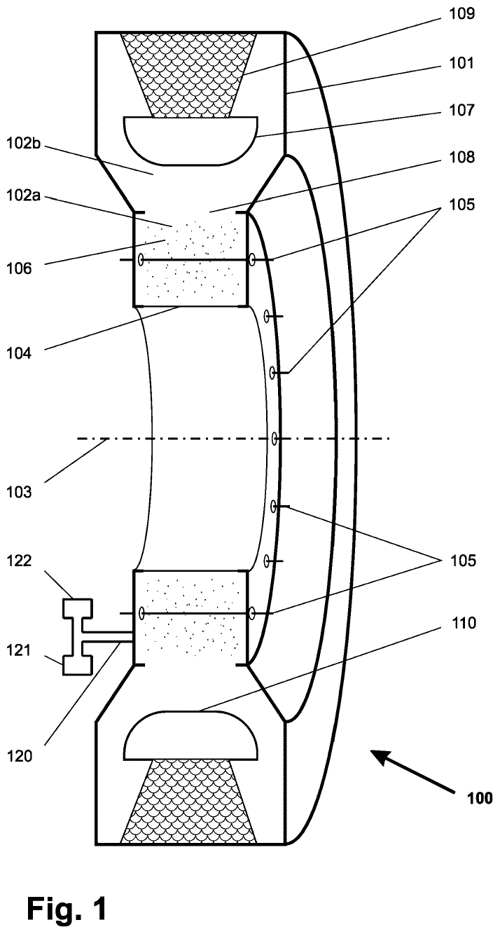

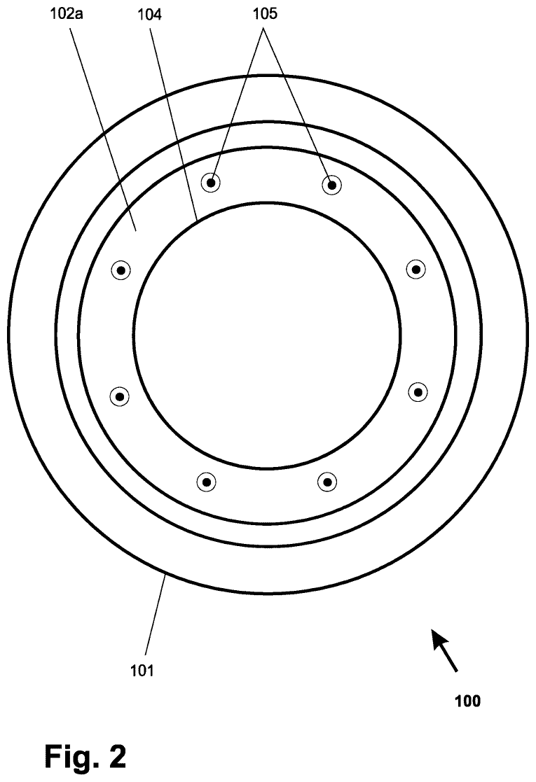

In FIG. 1 and FIG. 2, one and the same annular apparatus 100 according to the invention is shown schematically, where the apparatus 100 is shown in FIG. 1 in a perspective cross-sectional view and in FIG. 2 in a plan view. For better understanding, the terms “annular cylinder” and “annular disk” are defined here as they relate to an annular object. If the inner radius of a circular ring is subtracted from its outer radius, a measurement is obtained. If this measurement is less than the extension of the ring towards its ring axis, the ring is designed as an annular cylinder. However, if this measurement is greater than the extension of the ring in the direction of its ring axis, the ring is designed as an annular disk.

The annular apparatus 100 initially includes an annular housing 101 which delimits, at least in one region, an evacuable space divided into the evacuable spaces 102a and 102b. The evacuable space 102a is also referred to as the first evacuable space and the evacuable space 102b is also referred to as the second evacuable space. Due to the shape of the housing, this evacuable space is also annular and, in a preferred embodiment, has no walls dividing the evacuable space into annular segments, so that the evacuable space is continuous across the entire annular circumference. All components described below as belonging to the apparatus 100 and referred to as annular are radially symmetrical and have one and the same ring axis 103. On the inner ring side of the housing 101, the housing 101 is designed as an electron exit window 104 in the form of an annular cylinder, i.e., viewed in the exit direction of the electrons, the electron exit window 104 has a surface perpendicular oriented to the ring interior and, in the case of a circular annular cylinder such as the electron exit window 104, to the ring axis 103. A working gas contained in a first reservoir 121 is admitted into the evacuable space through at least one first inlet 120 in the housing 101, and a vacuum in the evacuable space in the range from 0.1 Pa to 20 Pa and preferably in the range from 1 Pa to 3 Pa is maintained by means of at least one pump device (not shown in FIG. 1). In FIG. 1, the at least one first inlet 120 is shown such that it opens into the evacuable space 102a. Alternatively and also preferably, the at least one first inlet opens into the evacuable space 102b.

An annular apparatus further has at least one first cathode and at least one first anode, between which a glow discharge plasma may be generated in the evacuable space by means of a first appliable electrical voltage provided by a first power supply device. According to the invention, in the apparatus 100 according to FIGS. 1 and 2, the housing 101 was designed as the first cathode, where the housing 101 designed as the first cathode may have, for example, the electronic ground potential of the apparatus 100.

The first anode of the apparatus 100, also referred to below as the electron beam generator 100, includes a number of wire-like electrodes 105 which extend through the evacuable space 102a and, in the case of a housing in the form of a circular ring, such as housing 101, are preferably arranged on an identical radius and at the same distance from one another around the ring axis 103. The wire-like electrodes 105, which may have a slightly positive voltage potential in a range of +0.25 kV to +5.0 kV relative to the housing 101, are passed through the housing 101 in an electrically insulated manner. Due to the electrical voltage applied between the wire-like electrodes 105 and the first cathode, a plasma is formed in the evacuable space 102a. The evacuable space 102a is therefore also referred to below as plasma space 102a.

An annular electron beam generator according to the invention further includes at least one second cathode and at least one second anode, between which a second electrical voltage is connected by means of a second power supply device. In the electron beam generator 100, a cathode 107 is formed as a second cathode and a screen-like anode 108 is formed as a second anode. Both cathode 107 and anode 108 have the shape of a ring.

In an annular electron beam generator according to the invention, the second cathode represents the cathode for emitting secondary electrons, which are subsequently accelerated, and for this purpose has a high-voltage electrical potential, preferably in the range of −100 kV to −300 kV. The second cathode 107 is electrically insulated from the housing 101 by means of an insulator 109.

In the electron beam generator 100 described in FIG. 1, the second anode 108 and the first cathode have the same electrical potential, which is designed as an electrical ground potential. Alternatively, the second anode and the first cathode may also have different electrical potentials.

By applying a high voltage potential in the range of −100 kV to −300 kV, positively charged ions are accelerated from the plasma 106 in the evacuable space 102a through the screen-like second anode 108 towards the second cathode 107. There, the ions strike a surface region 110 of the second cathode 107, the surface perpendicular of which is oriented to the annular interior of the housing, to the ring axis 103. When the ions strike the surface region 110, the ions have thus passed through a potential difference that largely corresponds to the acceleration voltage of the electron beam generator 100. When the ions strike, their kinetic energy is released in a very thin near-surface layer of the cathode 107 in the surface region 110, which leads to the release of secondary electrons. At the aforementioned electrical voltages on the second cathode 107, the ratio between released electrons and striking ions is on the order of magnitude of up to ten, which makes this type of generation of accelerated electrons very efficient. The resulting secondary electrons are strongly accelerated by the applied electric field and fly through the screen-like anode 108, which is designed in the form of an annular cylinder, and the plasma 106 in the space 102a. After traveling through the electron exit window 104, which may be designed as a thin metal film, for example, the electrons penetrate into the volume 114 enclosed by the annular housing 101 and in which a higher pressure may prevail than in the evacuable space and may be guided through the bulk material particles to be charged with electrons through the housing annular opening. All materials known from the prior art for an electron exit window, such as titanium, for example, may be used as material for the electron exit window 104. Furthermore, for the purpose of greater mechanical stability and cooling of the electron exit window 104, it is advantageous to provide the latter with a support screen, as is also known from the prior art.

It was previously described that secondary electrons are released when the ions strike the surface region 110 of the second cathode 107. In addition, however, material particles of the cathode are unfortunately also sputtered off, electrically non-conductive oxides being formed therefrom in a reactive process and then being deposited in the edge regions of the surface region 110 on the second cathode 107, causing undesirable arcs there, which in turn leads to process instabilities. The apparatus 100 according to the invention therefore also includes a second reservoir 122 in which a hydrocarbon-containing compound is contained and which compound, like the working gas, is also admitted into the evacuable space in a gaseous or vaporous state through the at least one first inlet 120. Preferably, for example, an alcohol as a hydrocarbon-containing compound may be admitted into the evacuable space. By admitting a hydrocarbon-containing compound into the evacuable space, carbon is also incorporated into the parasitic deposits on the second cathode 107 (as a result of cracking processes caused by the plasma and the beam electrons), which increases the electrical conductivity of the deposits, prevents surface creep discharges and thus reduces the tendency to arc.

Because the hydrocarbon-containing compound is to be admitted into the evacuable space in gaseous or vaporous form, it is advantageous to use a highly volatile hydrocarbon-containing compound for this purpose. From the group of alcohols, for example, methanol, propanol and especially ethanol are particularly suitable as hydrocarbon-containing compounds.

In order to admit the hydrocarbon-containing compound into the evacuable space, the hydrocarbon-containing compound may be present, for example, in liquid form in the second reservoir and the working gas, which is also to be admitted into the evacuable space, may be passed through the liquid hydrocarbon-containing compound in the second reservoir and then admitted into the evacuable space through the first inlet 120. In such an approach, vaporous components of the hydrocarbon-containing compound are introduced into the evacuable space with the working gas. Alternatively, the hydrocarbon-containing compound may also be admitted into the evacuable space by means of a separate mass flow controller.

With respect to the volume introduced into the evacuable space through the first inlet, it is advantageous when the hydrocarbon-containing compound has a proportion of approximately 0.5 to 10 percent by volume. The hydrocarbon-containing compound preferably has a proportion of about 2 to 6 percent by volume.

For the sake of completeness, it should be mentioned at this point that the annular electron beam generator 100 also has a device for cooling, as is also known from the prior art in apparatuses for generating accelerated electrons. For example, this device for cooling the electron beam generator 100 may include cooling channels that extend within the insulator 109 and through which a cooling medium flows.

The second anode 108, which in an annular electron beam generator is preferably designed as a screen-like annular cylinder segment and which represents the spatial limit between the evacuable spaces 102a and 102b, fulfills three essential tasks. Firstly, due to its voltage difference with respect to the second cathode 107, it causes an acceleration of the ions extracted from the plasma towards the second cathode. Secondly, it also causes an acceleration of the secondary electrons generated by the ion bombardment towards the electron exit window 104. Due to the fact that the screen structure of the second anode 108 is formed parallel to the secondary electron-emitting surface 110 of the second cathode 107, an electric field is formed such that the trajectories of the accelerated secondary electrons also run largely radially and antiparallel to the trajectories of the ions releasing them. Furthermore, the second anode 108 shields the plasma from the voltage potential of the second cathode 107, thereby preventing too many electrons from drifting away from the wire-like electrodes 111 and thus contributes to maintaining the plasma 106 in the evacuable space 102a.

As previously explained, the wire-like electrodes 105, which together constitute the first anode, are guided in an electrically insulated manner through the walls of the housing 101 and, in one embodiment, extend parallel to the ring axis 103 and completely through the evacuable space 102a. In an alternative embodiment, at least some of the wire-like electrodes 105 penetrate only one wall of the housing 101 and terminate within the evacuable space 102a without penetrating the opposite wall. Such wire-like electrodes 105 are also referred to below as stub anodes. By using stub anodes, it is possible to favorably influence the plasma formation in the evacuable space 102a and thus to increase the application area of the apparatus 100. In particular, when stub anodes are used, the tendency of the plasma to localize drops, and lower discharge currents, and thus also lower beam currents, may be controlled more reliably.

In a further embodiment, each wire-like electrode 105 is associated with a separate power supply device which provides the anode potential to the associated wire-like electrode 105. Alternatively, it is also possible for a power supply device to be associated with a plurality of wire-like electrodes 105, the power supply device providing the same anode potential for all associated wire-like electrodes 105. The electrodes associated with a power supply device may be, for example, adjacent wire-like electrodes 105. Alternatively, it is also possible for wire-like electrodes 105 that result from a sequence, such as every second wire-like electrode 105, every third wire-like electrode 105 or every fourth wire-like electrode 105, etc., to be associated with one power supply.

In a preferred embodiment, the apparatus 100 includes a power supply device having four independently controllable channels for providing the anode potential for the first anode. Each of the four channels of the power supply device is associated with a different 90° annular sector of the annular space 102a, where all wire-like electrodes 105 of a 90° annular sector are electrically conductively connected to the associated channel of the power supply device, so that all wire-like electrodes 105 of a 90° annular sector have one and the same anode potential. Alternatively, the power supply device having four channels may also be replaced by four separate power supply devices or by two power supply devices, each having two separately controllable channels. With such a circuit configuration for the wire-like electrodes 105, the four 90° annular sectors may be controlled separately for the purpose of plasma formation. The respective discharge current in the individual annular sectors and, as a result, the electron current or the treatment dose in the corresponding circumferential regions of the cathode or the atmosphere-side treatment chamber may then be controlled so that a temporally well-defined course, which is also spatially well-defined across the entire circumference of the electron exit window, is obtained (the course being homogeneous or adjustable as a function of the ring angle depending on the material being treated and technology requirements).

In a power supply device having four channels, the individual channels of the power supply device may either be operated together but with a freely programmable current setpoint, or, alternatively, they may be operated one after the other, where the sequence of operation, the duration of operation and the temporal offset of the individual channels are freely programmable.

For example, the four 90° annular sectors may all be activated simultaneously. The local discharge current strength in the individual sectors may be selected to be the same or different, depending on requirements. However, it has proven problematic to supply all four 90° annular sectors simultaneously with the energy required to form a stable plasma. Preferably, therefore, the power supply device having the four channels is configured such that only one channel or a group of two or three channels is active at any one time, and the anode voltage is provided for the wire-like electrodes 105 of the associated 90° annular sectors, and such that the 90° annular sectors or groups are activated one after the other. Adjacent 90° annular sectors do not necessarily have to be activated immediately one after the other. It may also be useful to activate opposite 90° annular sectors one after the other. This may then also be done with either identical or different discharge current strengths as desired (or depending on the technology).

In an advantageous embodiment, the discharge current intensity may be set equally in each 90° annular sector or each group, which minimizes the fluctuation in the current demand from the associated high-voltage device. The regulation of an electron current density and treatment dose that is homogeneous in the temporal mean over the circumference or that varies depending on the angle may be achieved by means of an individual duty cycle of the 90° annular sectors or groups. For a ready ignition of plasma and for the most uniform current demand from the high-voltage device, it is also advantageous to provide a freely programmable temporal offset between switching off the previous 90° annular sector (or group) and switching on the next.

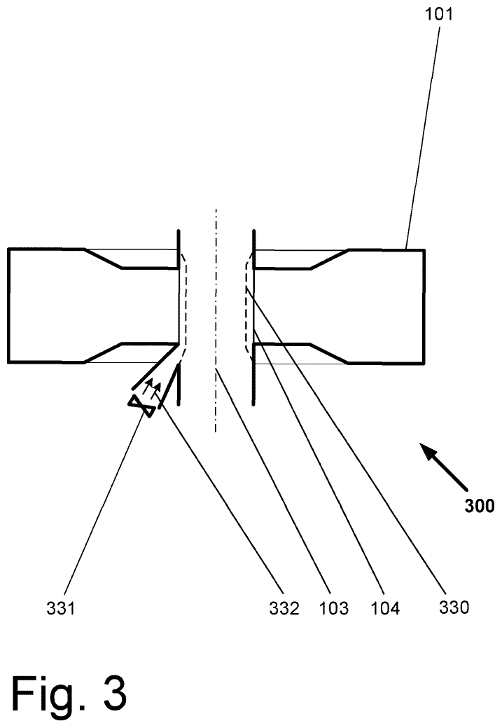

FIG. 3 shows the schematic sectional view of a first alternative apparatus 300 according to the invention. The basis of the apparatus 300 is an annular apparatus as described in FIGS. 1 and 2. Therefore, the apparatus 300 also includes an annular housing 101, in which the ring axis 103 is oriented perpendicularly or at an angle of up to 10° from the perpendicular, so that, for example, bulk material to be treated with accelerated electrons may fall from above to below through the annular opening of the annular housing 101. A cylindrical protective screen 330 is arranged within the annular opening in front of the electron exit window 104 and mechanically protects the electron exit window from falling bulk material particles. The protective screen 330 may consist of a heat-resistant gauze material, for example.

A flow 332 of a gaseous medium is formed by means of a first device 331 and is directed from below to above between the electron exit window 104 and the protective screen 330. On the one hand, this flow 332 cools the electron exit window 104 and, on the other hand, the gaseous medium, which simultaneously also penetrates through the protective screen 330, slows the fall of the bulk material particles, increasing the mean dwell time of the bulk material particles in front of the electron exit window 104.

In addition, by additionally introducing the gaseous medium in the region between the electron exit window 104 and the protective screen 330, dirt particles which fall downwards with the bulk material particles and which are smaller than the openings of the protective screen 330 are kept away from the electron window 314, thereby reducing its contamination and thus increasing its service life.

In a further embodiment of the invention, the apparatus 300 has at least one sensor by means of which first actual values that represent the velocity of fall of the bulk material particles in front of the electron exit window 104 are detected. Within a first evaluation device, these actual values are compared to a first target value for the velocity of fall of the bulk material particles and a first comparison value is generated and the intensity of the flow 332 of the gaseous medium is regulated as a function of the first comparison value. The intensity of the flow 332 of the gaseous medium may be changed, for example, by changing the power of the first device 331 by means of a control device. In this way, it may be ensured that the bulk material particles are subjected to a homogeneous and/or predetermined dose of accelerated electrons.

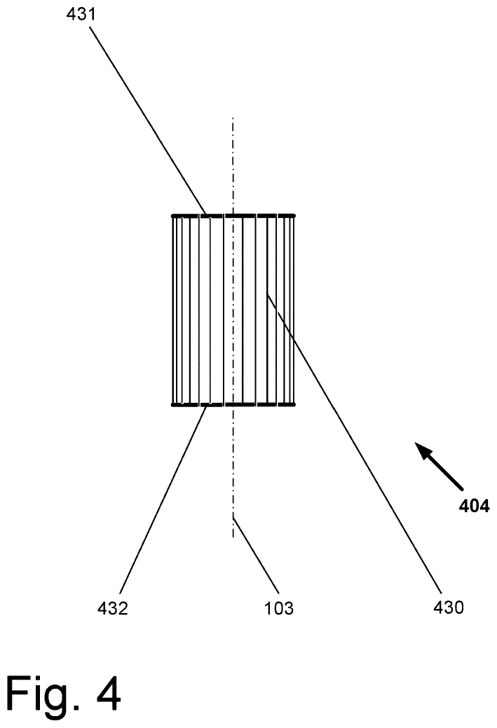

It has already been described above that the protective screen in front of the electron exit window of an apparatus according to the invention may consist, for example, of a heat-resistant gauze material. Such a gauze material usually includes horizontally and vertically running metal threads or metal wires. FIG. 4 schematically shows an alternative protective screen 404 that, at least at the height of the electron exit window 104, includes only vertically running metal first wires 430 which are stretched between an upper connecting ring 431 and a lower connecting ring 432 and where respective adjacent first wires 430 are spaced from one another at a distance that is smaller than the diameter of the bulk material particles. The second wires 430 thus also run parallel to the ring axis 103 of the annular electron beam generator. Horizontally running wires of a protective screen in front of an electron exit window have the disadvantage that bulk material particles passing by the protective screen from above to below introduce impact energy into the horizontally running wires, which may lead to deformation of or damage to a protective screen. With a protective screen 404 according to FIG. 4, which has only vertically running first wires 430, there is no longer such a source for error. Such a protective screen 404 may be used in all embodiments of apparatuses according to the invention described above and below.

In one embodiment, the metal first wires 430 are formed individually or electrically insulated from one another in groups. By means of at least one measuring device, the electrical current flowing through a respective first wire 430 or a first group of first wires (430) or a second actual value representing this electrical current may be detected and is forwarded to a second evaluation device. This second actual value is compared within the second evaluation device to a second target value for the electrical current, resulting in a second comparison value. Depending on the second comparison value, the cathode current of the annular electron beam generator in the associated 90° annular segment may then be controlled by means of the discharge current intensity and/or the discharge duration. In this way, quality control may be carried out to determine whether or not the application of accelerated electrons is homogeneous in all annular segments or occurs according to a predetermined angular distribution across the circumference and with the required irradiation intensity. Depending on the second comparison value, the cathode current of the annular electron beam generator may thus be controlled by means of the discharge current intensity and/or the discharge duration in the individual 90° annular segments or groups.

By directly measuring the electron current density in the treatment space with temporal and spatial resolution, the uncertainty described above (due to an unknown or locally or temporally changing Townsend's second ionization coefficient γ) may be eliminated and a treatment dose that conforms to the technology may be ensured.

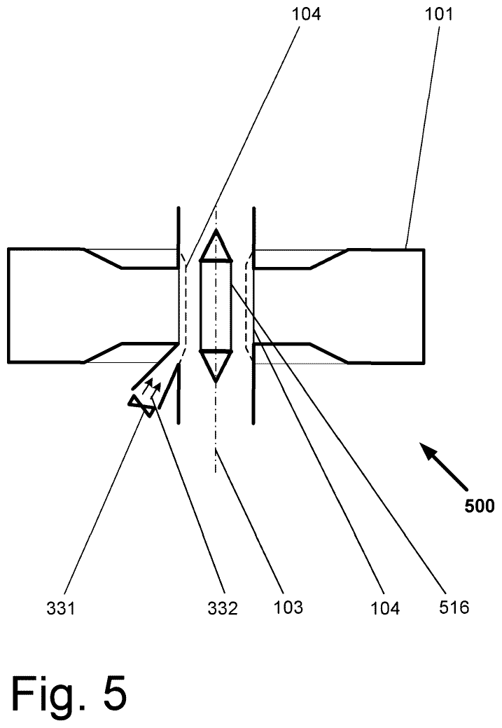

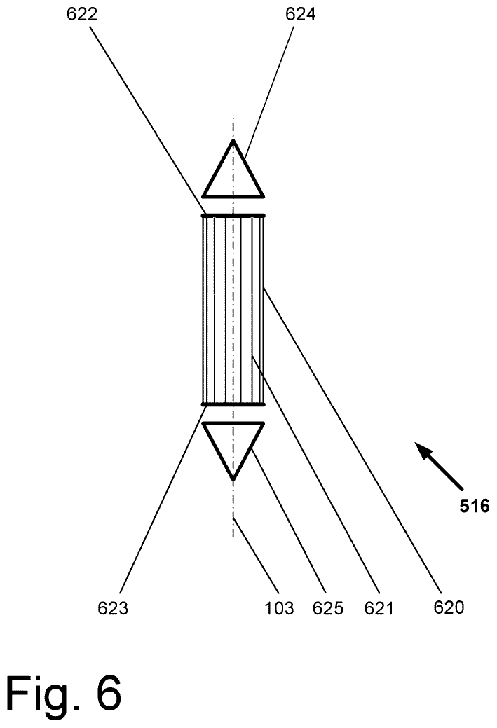

In FIG. 5, a second alternative apparatus 500 according to the invention is shown schematically in section, and initially has all the elements and features of the apparatus 300 from FIG. 3. In addition, the apparatus 500 includes a rotationally symmetrical element 516, the axis of rotation of which is identical to the ring axis 103 of the annular apparatus 100 and the annular apparatus 300. The rotationally symmetrical element 516 is shown schematically in greater detail in an exploded view in FIG. 6.

The rotationally symmetrical element 516 initially has a base body 620 in the form of a hollow cylinder, where the outer surface of the hollow cylinder consists of metal second wires 621 which are stretched between an upper connecting element 622 and a lower connecting element 623 and which run parallel to the ring axis 103 of the annular electron beam generator. Adjacent second wires 621 are each spaced apart by a distance smaller than the diameter of the bulk material particles that fall through the annular opening, so that no bulk material particles may travel into the interior of the base body 620. The upper connecting element 622 and the lower connecting element 623 may be disk-shaped or annular, for example. The rotationally symmetrical element 516 further includes an upper closure element 624 and a lower closure element 625, which may both be conical, for example, and in which the conical outer surface is preferably completely closed. Alternatively, the two closure elements 624, 625 may also have a different shape that promotes the flow of the gaseous medium. In an embodiment, the rotationally symmetrical element 516 is arranged within the annular electron beam generator and dimensioned such that the metal second wires 621 extend at least across the height of the electron exit window 104.

Two positive effects are essentially achieved with such a rotationally symmetrical element 516. On the one hand, the bulk material particles are guided into an annular gap between the protective screen 104 and the base body 620 of the rotationally symmetrical element 516, as a result of which a thin annular curtain of bulk material particles is formed and guided past the electron exit window 104, which leads to a good result with regard to subjecting the bulk material particles to accelerated electrons. On the other hand, the back side of bulk material particles may also be subjected to the primary electrons, which succeed in penetrating the curtain of bulk material particles and the not completely closed base body 620 of the rotationally symmetrical element 516 from the opposite side of the electron exit window, further improving the result of subjecting the bulk material particles to accelerated electrons. Such a rotationally symmetrical element 516, as described here only by way of example for apparatus 500, may alternatively also be used in all other embodiments of apparatuses according to the invention.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. An annular apparatus for generating accelerated electrons, comprising an annular housing which delimits an annular evacuable space and has an annular electron exit window; at least one first reservoir containing a working gas; at least one first inlet for supplying the working gas from the first reservoir into the annular evacuable space; at least one first cathode and at least one first anode, between which a glow discharge plasma may be generated in the annular evacuable space by a first applied electrical voltage, wherein ions from the glow discharge plasma may be accelerated onto the surface of an annular second cathode and electrons emitted by the annular second cathode may be accelerated towards the annular electron exit window by a second electrical voltage applied between the annular second cathode and an annular second anode, wherein the housing is designed as a first cathode; wherein the first anode comprises a number of wire-like electrodes which extend completely or partially through the annular evacuable space, wherein a second reservoir contains a hydrocarbon-containing compound which may be admitted into the evacuable space through the at least one first inlet.

2. The apparatus of claim 1, wherein the wire-like electrodes are arranged within the annular evacuable space in a ring shape about the ring axis of the annular housing and run parallel to the ring axis.

3. The apparatus of claim 1, wherein the ring axis of the annular housing is oriented perpendicularly or at an angle of up to 10° from the perpendicular.

4. The apparatus of claim 1, wherein a cylindrical protective screen is arranged in front of the electron exit window.

5. The apparatus of claim 4, further comprising a protective screen which, at least in the region in front of the electron exit window, comprises only vertically running first wires.

6. The apparatus of claim 5, wherein the vertically running first wires are formed electrically insulated from one another in groups; the electrical current flowing through one of the vertically running first wires or a group of the first wires of the protective screen may be detected by at least one measuring device; the second actual value may be compared to a second target value for the electrical current and a second comparison value may be generated by a second evaluation device; the cathode current of the annular electron beam generator in the associated 90° annular segment may be controlled as a function of the second comparison value.

7. The apparatus of claim 3, further comprising a first device with which a flow of a gaseous medium directed from below to above may be generated within the annular opening of the annular housing.

8. The apparatus of claim 7, further comprising at least one sensor by which a first actual value, which represents the velocity of fall of bulk material particles to be treated with accelerated electrons in front of the electron exit window, may be detected; a first evaluation device by which the first actual value may be compared to a first target value for the velocity of fall of the bulk material particles and a first comparison value may be generated, and a control device by which the power of the first device may be regulated as a function of the first comparison value.

9. The apparatus of claim 3, further comprising a rotationally symmetrical device, the axis of rotation of which is identical to the ring axis, comprising a base body in the form of a hollow cylinder, wherein the outer surface of the hollow cylinder consists of metal second wires running parallel to the ring axis.

10. The apparatus of claim 1, further comprising a power supply device having four independently controllable channels for providing the anode potential for the first anode, wherein each of the four channels of the power supply device is assigned to a different 90° annular sector of the annular space 102a and wherein all wire-like electrodes of a 90° annular sector are electrically conductively connected to the associated channel of the power supply device, so that all wire-like electrodes of a 90° annular sector have one and the same anode potential.

11. The apparatus of claim 10, wherein the channels of the power supply device are operable together but with a freely programmable current setpoint.

12. The apparatus of claim 10, wherein the channels of the power supply device are operable one after the other but with a freely programmable sequence, duration and programmable time offset.
